(19) United States
(12) Patent Application Publication
(10) Pub. No.: US 2011/0227237 A1
(43) Pub. Date: Sep. 22, 2011

(54) RESPIRATORY GAS HUMIDIFIER FOR USE IN MR TOMOGRAPHY

(76) Inventor: Torsten Hertz, Lubeck (DE)

(21) Appl. No.: 12/734,642

(22) PCT Filed: Nov. 13, 2008
(86) PCT No.: PCT/EP2008/009600

§ 371 Date: May 27, 2011

(30) Foreign Application Priority Data
Nov. 13, 2007 (DE) 20 2007 015 930.7

(51) Int. Cl. A61M 16/16; A61M 16/10

(52) U.S. Cl. 261/129

(57) ABSTRACT

The invention relates to a respiratory gas humidifier for heating and humidifying respiratory air, having a humidifier module through which water and respiratory air flow, the water and respiratory air flows being separated by flat elements that are permeable to water vapor, said respiratory gas humidifier having a fluid pump and a heating element for the water, characterized in that the fluid pump is a piezo pumps.

### BACKGROUND

The invention relates to a respiratory gas humidifier for heating and humidifying respiratory air for use in combination with transport- and/or intensive care ventilators. More particularly, the invention also relates to a respiratory gas humidifier suitable for use in MRI scanners.

Ventilators for adults or newborns are supplied with pressurized gas (oxygen and pressurized air) from flasks or via the wall connectors in the ward. This pressurized gas is dry and also cold after relaxing from line pressure to ventilation pressure. Since dry, cold air from the ventilator would damage the lungs of the patients and leads to additional heat losses, respiratory gas humidifiers are used in intensive care to allow the air to be heated and humidified to physiological conditions.

A concept for humidifying and heating the respiratory gasses, very widely used in the past, is the cooker principle, in which the respiratory gas flow is guided over heated water. An electric heating ensures that the water is heated in the process. Temperature sensors monitor the system in a closed-loop.

In a further concept, an electric liquid-pump (direct-current drive) guides water through hollow-fiber elements that are heated by alternating-current heating elements. In the process, warm water flows around the outside and respiratory air flows around the inside of these fibers. The pump operates at a constant rotational speed in order to maintain the volume flow.

In any case, the conversion of electric into kinetic energy is based on magnetic fields varying in time and/or space. It is unsurprising that this drive concept reaches its limits in the vicinity of strong external magnetic fields, if the useful field of the motor is superposed with the external interference-field and the required continuous rotational movement is interfered with or prevented. However, this is precisely the case when a respiratory gas humidifier is introduced into the homogeneous field of an MRI magnet (magnet of a magnetic resonance imaging scanner or nuclear magnetic resonance scanner). Furthermore, the closed-loop control electronics are not suitable for use in the vicinity of the radio-frequency fields of a magnetic resonance imaging scanner.

Known technical approaches for the application in magnetic resonance imaging scanners are limited to e.g. the use of magnetic shielding for the motor and the electronics and to spatial displacement of the motor to a site with a reduced interference-field strength, that is to say as far away as possible from the homogeneous field and the scattered field of the magnet in order to attempt to avoid these problems. However, none of these measures offers satisfactory results for humidifying the respiratory gasses for newborns and adults in respect of:
1. MRI magnetic field strengths above 1.5 Tesla
2. Adverse effects on MR imaging
3. Achieving physiological respiratory humidity and respiratory gas temperature
4. Transport of incubator and respiratory gas humidifier, if these are designed as separate and spaced apart items of equipment in order to avoid the aforementioned interferences.

An object is developing a closed-loop controlled air humidification and heating for ventilators, in which the problems of the magnetic components and the problems of the electrical interference are avoided.

### SUMMARY

Magnetic components are dispensed with entirely and a piezo-pump is used for circulating the water.

The known piezoelectric effect is based on the displacement of the spatial crystal structure of certain crystals and ceramics when an electric potential is applied. It is used industrially and commercially, particularly for fast and precise positioning or as a linear drive for relatively slow processes.

The use as a pump could not be implemented for a long time due to limited lifespan, poor degree of efficiency and too low delivery amounts. The linear movement of the piezo-element was usually converted into rotational movement for motors by means of mechanical elements.

It was now discovered that the piezoelectric effect can surprisingly be used for driving the liquid pump of a respiratory gas humidifier.

Newest developments in materials and technologies could mitigate all the above limitations. An industrially available piezo-pump according to the so-called ultrasound principle is provided by Nitto Kohki/Japan. Integrated electronics with this functional principle ensure a constant number of strokes for a given mains frequency and thus a constant delivery volume per minute.

It was now discovered that such a piezo-pump can advantageously be used in particular as a liquid pump in equipment that should be inserted into the magnetic field of an MRI scanner.

A feature of one embodiment is the integration of hollow-fiber elements for air humidification, water heating, tube heating, the piezo-pump and the sensors (pressure, temperature and lack of water) as a medical product for which the use in MRI and the combination with ventilators and anesthesia equipment e.g. for adults or children and for the combination with ventilators for newborns and an MRI incubator whilst at the same time maintaining both the MRI compatibility and the constancy of the physiological temperature and humidity of the respiratory gas.

### BRIEF DESCRIPTION OF THE DRAWINGS

In the following text, the functional design and demarcations and differences from conventional respiratory gas humidifiers are explained in an exemplary fashion on the basis of advantageous embodiments with reference to the attached drawings, in which:

FIG. 1 shows the principle of the humidifier in a block diagram (air and water flows);

FIG. 2 shows the basic design of an advantageous embodiment in conjunction with an MRI incubator;

FIG. 3 shows the basic design of an advantageous embodiment as stand-alone equipment;

FIG. 4 shows an electric circuit with source and drain;

FIG. 5 shows the circuit of FIG. 4 with a blocking filter;

FIG. 6 shows the frequency response of the blocking filter of FIG. 5;

FIG. 7a shows the support suitable for attaching the respiratory gas humidifier to the MRI incubator;

FIG. 7b shows the attachment of the ventilator on the support and the mounting of this arrangement on the MRI incubator;

FIG. 7c shows the attachment of the respiratory gas humidifier on the support;

FIG. 8 shows the display and user interface of the humidifier integrated into the operating field of the incubator;

FIG. 9 shows the stand-alone design of the humidifier; and

FIG. 10 shows how the electronic closed-loop control and monitoring device is embedded into the humidifier system.

### DETAILED DESCRIPTION

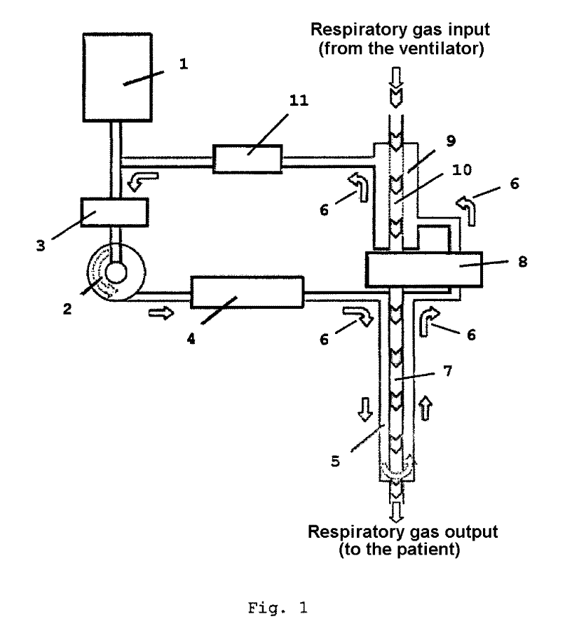

The advantageous embodiment of the invention shown in FIG. 1 has a water reservoir 1, which contains a water supply for heating and humidifying the respiratory air.

This water is passed through the circuit by a liquid pump 2 that is designed as a piezo-pump. For this, a temperature measuring device 3 first of all measures the temperature of the circulating water and water from the water reservoir 1 is added when water is used. Downstream of the piezo-pump 2, the water passes through a heat exchanger 4, within which it is warmed by electrical means. It subsequently passes through a multi-lumen tube 5, in which the water circulates in the direction of the arrows 6 on the outside and in doing so heats the respiratory gas flowing through the inner lumen 7. The water and the respiratory gas flow through a measuring device 8 for water—and respiratory gas pressure that, together with the other electronic components, is connected to a closed loop (not illustrated in FIG. 1). After passing through the measuring device 8, the water flows through a humidifying module 9 and flows around a pipe or a tube 10 that is permeable to water vapor and through which respiratory gas flows, the latter being humidified as a result thereof. During the continuous throughput through the circuit, a device 11 for recognizing air bubbles monitors whether the system still has sufficient water.

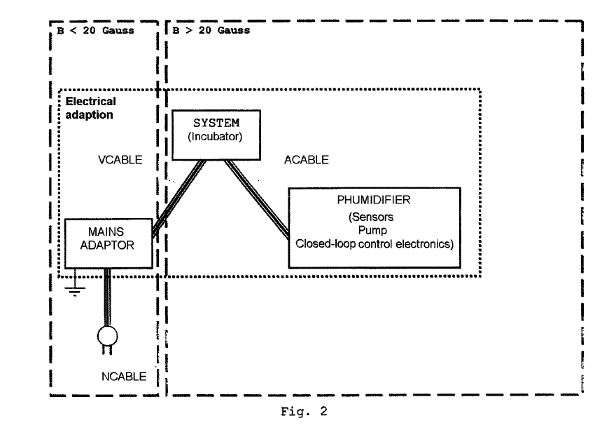

From the point of view of the electric properties, the MRI compatibility is generally achieved by a spatial separation of the magnetically sensitive voltage-supply unit (MAINS ADAPTOR in FIG. 2) of the non-magnetic piezo-pump (contained in PHUMIDIFIER) and by the suitable screening of the electronic and electric components. The connection cables required for this have been screened and earthed, and the signals passed therein are filtered. These measures are explained in detail below.

The system control SYSTEM of the incubator serves for entering the target value and displaying the actual values and possible alarm notices.

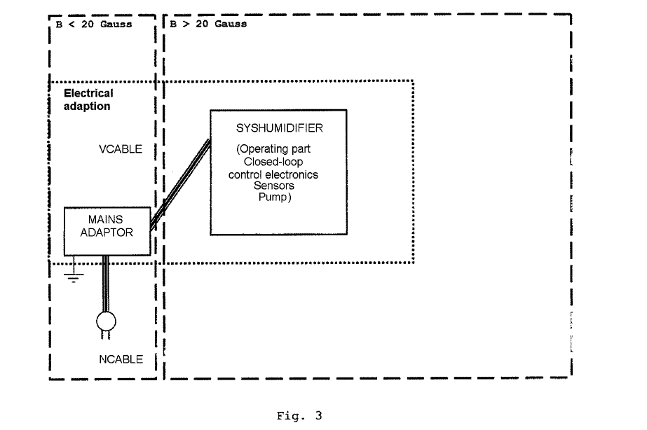

However, the respiratory gas humidifier according to the invention can also be used for different purposes; FIG. 3 illustrates the stand-alone version. In this case, the operating part is attached directly to the respiratory gas humidifier (SYSHUMIDIFIER) and not to the incubator or a further control device.

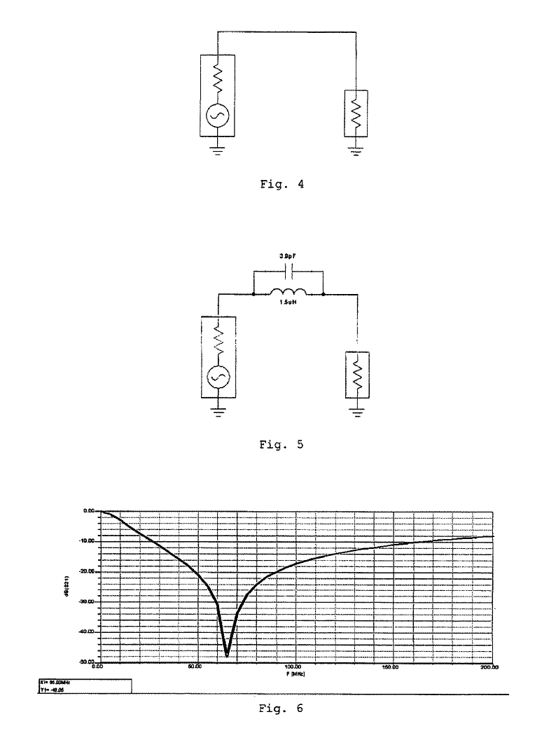

Achieving electrical adaptation
i. Screening: As a basic measure, all components involved and the connections thereof are screened.
ii. Earthing: All surrounding screening ends at one earthing point; the presence of earthed loops adversely affects the imaging and should therefore be avoided at all costs.
iii. Filtering: As third and most important measure, the signals between MAINS ADAPTOR, SYSTEM and PHUMIDIFIER are filtered.
   Generally: Every signal is passed along a path (generally an electrical cable) between source (Q) and drain (S). See FIG. 2. The source is illustrated on the left-hand side and the drain is illustrated on the right-hand side. The frequency spectra applied by the MRI scanner are narrowband spectra in the respective equipment class and so the introduction of a selective blocking filter of higher order along each signal path between MAINS ADAPTOR, SYSTEM and PHUMIDIFIER minimizes the aforementioned interferences.
   Such a blocking filter can easily be implemented as an LC circuit (2nd order passive filter), as shown in FIG. 3. In the case of piezo-technology, it additionally is advantageous that the useful frequency range of the pump (50 Hz or 60 Hz) utilized in this application is far enough away from that of the MRI scanner (42-300 MHz) and so the filtering does not cause side effects. FIG. 4 shows the frequency response when the blocking filter according to FIG. 3 is used. The resonance frequency was matched to an MRI system with a 1.5 T magnetic field strength corresponding to a Larmor frequency of 63.9 MHz. In this region, the introduced damping is better than 40 dB. This filtering should now be applied to each of the aforementioned signal paths at both ends of VCABLE and on the SYSTEM-side end of ACABLE.

NCABLE: Screened mains connection cable (3-wired: L, N, PE) with a length of approximately 30 cm (already present in FIG. 2 because it is required as an AC supply cable for the incubator).

MAINS ADAPTOR: Screened box containing the switched-mode power supply. It is spatially separate from the rest of the equipment and is operated in a region in which the remaining scattered field from the MRI scanner is very weak (flux density in the air B<20 Gauss). Hence there are no adverse effects on the functioning of the switched-mode power supply (already present in FIG. 2 because it is required as a DC voltage generator (12 V) for the incubator).

PCONTROL: Control electronics for the respiratory gas humidifier. These electronics are supplied with 5 V directly from the mains adaptor. The outputs via VCABLE are the power signals for the heat exchanger; the inputs are the signals from the temperature sensors. All lines from the input and output are provided with a blocking filter according to FIG. 3.

VCABLE: Screened connection cable that contains the lines described with respect to MAINS ADAPTOR. This cable is long enough (L=4.5 m) for obtaining the spatial separation of the MAINS ADAPTOR from the equipment.

PHUMIDIFIER: The humidifying unit. It accepts the power signals from the MAINS ADAPTOR and the control signals from the SYSTEM and returns the measured values back to the SYSTEM.

ACABLE: Screened connection cable from the respiratory gas humidifier to the SYSTEM. It passes the power signals and the actual values from the sensors (temperature) and error messages back to the SYSTEM.

SYSTEM: The system electronics of the incubator. The signals arriving from the PHUMIDIFIER are evaluated and displayed; the user is notified by the equipment in the case of deviations.

Moreover, the mains voltage and the DC voltage are passed through PHUMIDIFIER and mains adaptor and are each provided with blocking filters as per FIG. 3.

### Achieving the Mechanical Adaptation

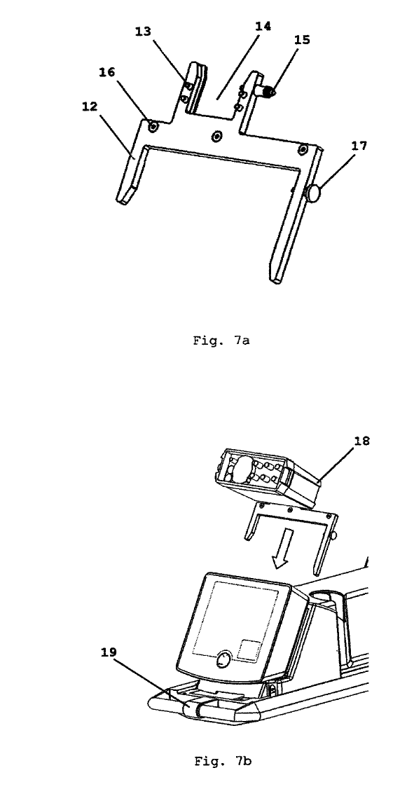

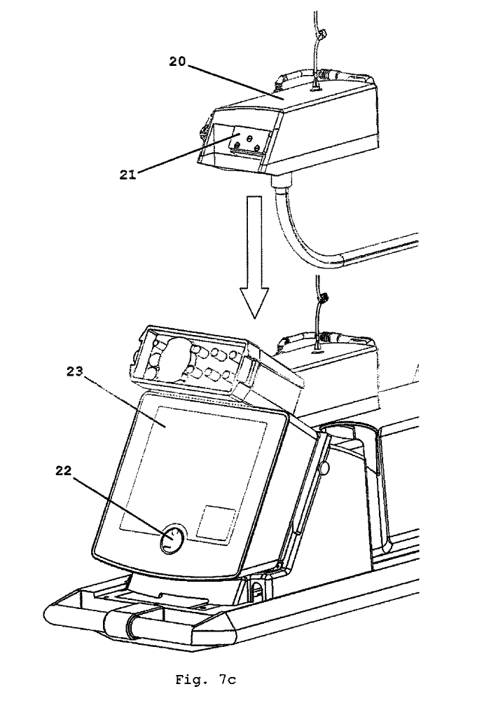

The drawings in FIG. 7 show, step by step, the adaptation of the respiratory gas humidifier to the MRI incubator. The basic connection element in FIG. 7a is the support 12 made of the non-magnetic material aluminum. This support has a fork-like geometry so that it can be placed onto the operating part of the incubator 19 from above in a suitable fashion (FIG. 7b). Here, the knurled screw 17 serves for the attachment. The three bolts 16 serve for the locking in the end position. The four attachment screws 13 allow the ventilator 18 to be attached from the rear side of the support. The cut-out 14 in the support holds the front-side mounting plate 21 of the respiratory gas humidifier 20 in an interlocking fashion (FIG. 7c); a force fit is obtained by the spring-pretensioned tension-bolt bar 15 and the fitting bore in 21. Accordingly, it is also fitted from above in this case; pulling 15 can initiate the disassembly of the humidifier 20 without additional tools, e.g. for an intended post-processing, cleaning or if the humidifier should not be used for the application or examination.

The connection cable (ACABLE in FIG. 2) between humidifier and incubator required for supply and control is not illustrated in FIG. 7 for reasons of clarity.

### Achieving the Interaction With the User

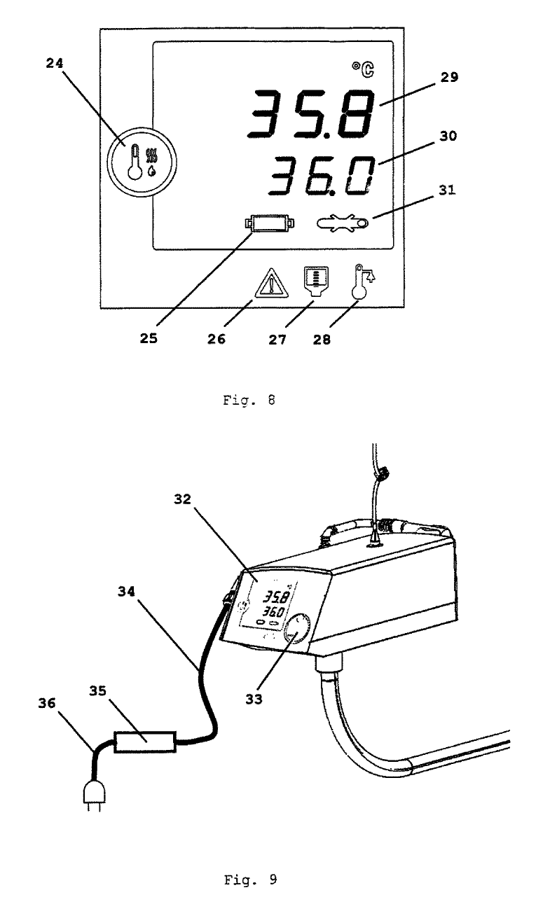

The general system sketched out in FIG. 1 is extended by FIG. 8 in respect of the input and output elements interacting with the user. In the first case—belonging to FIG. 2—these elements are embedded in the already present operating field 23 of the incubator.

Here, the numeric display 29 is used for displaying the actual temperature of the heated respiratory gas. The display 30 displays the active target temperature set by the user. A change in this target temperature is brought about by actuating the push-button 24; the changing (rotating) and confirming (pushing) of the new set value is achieved using the rotary encoder 22 already present in the incubator.

The signal light 25 shows that the heating closed-loop is active. The remaining indicators indicate alarm states of the humidifier: the signal light 26 is active in the context of those alarms for which the corresponding sections in the operating manual should be observed; the signal light 27 indicates an empty water reservoir; the signal light 28 indicates that the actual temperature is too high in relation to the target value; the signal light 31 indicates bent tubes or other obstructions.

All alarms are also emitted acoustically by audio warning.

In the second case, FIG. 9 shows the stand-alone design of the respiratory gas humidifier, the arrangement corresponding to the systematic design in FIG. 3. The front side of said respiratory gas humidifier is provided with an operating field as per FIG. 8, complemented by the rotary encoder 33. The operating and alarm concept precisely corresponds to that of the variant embedded in the incubator. For the energy supply, use is made of a sufficiently long feed line 34 (corresponding to VCABLE in FIG. 3). The spatially separate mains adaptor 35 (corresponding to MAINS ADAPTOR in FIG. 3 and at a distance of at least 2.5 m from the MRI magnet) and the mains cable with connector 26 (corresponding to NCABLE in FIG. 3) complete the supply path.

### Achieving the Closed-Loop Controlled Respiratory Gas Temperature

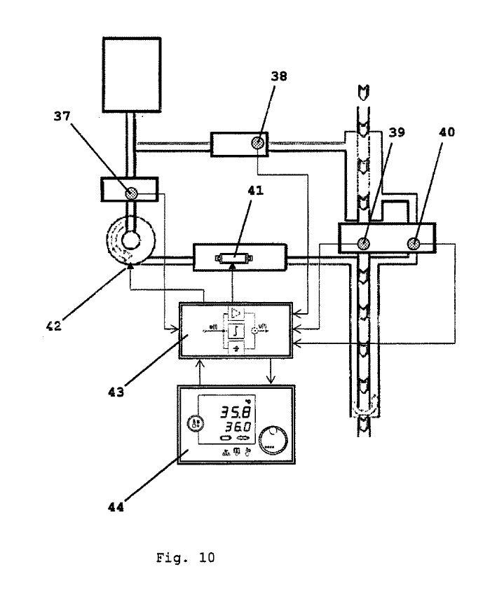

In FIG. 10, the block diagram described in FIG. 1 is extended by the required elements for temperature closed-loop control. These can be approximately subdivided into the categories: sensor, closed-loop controller and actuators.

Sensors:

The measurement pick-up for the actual value of the temperature is the thermistor (NTC) 37.

The electronic pressure sensor 39 measures the pressure in the respiratory air path.

The electronic pressure sensor 40 measures the pressure in the water circuit.

The optical sensor 38 detects air bubbles (which correspond to an insufficiently filled water circuit or lack of water), which sensor is implemented as a photo-sensor applied to the transparent tube piece.

Actuators:

The variable for the closed-loop controller output is the pulse-width modulated (PWM) mains voltage (0 . . . 100%), which is applied to the heating element 41 and outputs a corresponding thermal power (0 . . . 150 W) to the heat exchanger.

The piezo-pump 42 can be switched on or off in order to carry out maneuvers such as filling or rinsing the humidifier.

Closed-Loop Controller:

The closed-loop control device 43 calculates the variable (PWM) for the heating 43 from the difference between the actual and target values by means of a PID algorithm. Additionally, alarm conditions are calculated from the additional sensors 38 (air bubbles in the water circuit), 39 and 40 (pressure increase as a result of a bent tube) and the linearization of the thermistor characteristic line. The user interface 44 serves as a source (target value prescription) and drain (measured value display, alarm display).

1. A respiratory gas humidifier for heating and humidifying respiratory air with a humidifying module though which water and the respiratory air flow and in which the water- and respiratory air flows are separated by water-vapor permeable planar elements, with a liquid pump and a heating for the water, characterized in that the liquid pump is a piezo-pump, in that the humidifying module has a hollow-fiber element, in that it has a multi-lumen tube for separate guidance of the respiratory gas and the water to the tube heating, arranged upstream of the liquid pump, a temperature measuring device for the water carried in the circuit pressure sensors for the water pressure and respiratory gas pressure.

2. (canceled)

3. (canceled)

4. The respiratory gas humidifier as claimed in claim 1, characterized in that it contains integrated sensors for temperature and pressure and has closed-loop control devices.

5. The respiratory gas humidifier as claimed in claim 1, characterized in that it contains an optical sensor for monitoring lack of water.

6. The respiratory gas humidifier as claimed in claim 1, characterized in that the equipment when combined with the MRI incubator is supplied with electricity via the incubator and the incubator contains the display and operating elements within the operating electronics of the incubator.

7. The respiratory gas humidifier as claimed in claim 1, characterized in that it contains attachment regions and attachment elements that are combined with and suitable for the incubators.

8. The respiratory gas humidifier as claimed in claim 1, characterized in that the equipment contains its own display and a separate electricity supply.

9. (canceled)

10. (canceled)

11. The respiratory gas humidifier as claimed in claim 1, characterized in that it has alarm devices for reporting malfunctions.

12. The respiratory gas humidifier as claimed in claim 4, characterized in that it contains an optical sensor for monitoring lack of water.

13. The respiratory gas humidifier as claimed in claim 4, characterized in that the equipment when combined with the MRI incubator is supplied with electricity via the incubator and the incubator contains the display and operating elements within the operating electronics of the incubator.

14. The respiratory gas humidifier as claimed in claim 4, characterized in that it contains attachment regions and attachment elements that are combined with and suitable for the incubators.

15. The respiratory gas humidifier as claimed in claim 4, characterized in that the equipment contains its own display and a separate electricity supply.

16. The respiratory gas humidifier as claimed in claim 4, characterized in that it has alarm devices for reporting malfunctions.
